(19) United States
(12) Patent Application Publication
(10) Pub. No.: US 2009/0299491 A1
(43) Pub. Date: Dec. 3, 2009

(54) Modular shock absorbers for prosthetic limbs

(76) Inventors: Tracy C. Slemker, Clayton, OH (US); Scott R. Schall, Englewood, OH (US); Lucinda Busch, Clayton, OH (US); Steven Steinbarger, Wilmington, OH (US); Robert Hoskins, Springboro, OH (US); Paul L. Galloway, Clayton, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 12/455,675

(22) Filed: Jun. 4, 2009

Related U.S. Application Data
(63) Continuation-in-part of application No. 11/069,832, filed on Mar. 1, 2005.
(60) Provisional application No. 60/549,115, filed on Mar. 1, 2004.

(51) Int. Cl. A61F 2/78; A61F 2/74

(52) U.S. Cl. 623/33; 623/27

(57) ABSTRACT

A modular shock absorber for a prosthetic limb which may be selectively interconnected between a socket for receiving a residual limb and other prosthetic limb components. An exemplary modular shock absorber includes a generally C-shaped body having a substantially planar first extension coupled to a prosthetic socket, a substantially planar second extension coupled to a prosthetic knee chassis, a substantially curved closed end connecting the first extension and the second extension, and a bias interposing the first extension and the second extension. Some exemplary modular shock absorbers may include first and second extensions that are horizontally and/or angularly offset relative to each other.

### CROSS REFERENCE TO RELATED APPLICATIONS

This application is a continuation-in-part of prior application Ser. No. 11/069,832, filed Mar. 1, 2005, which claims the benefit of provisional Application No. 60/549,115, filed Mar. 1, 2004, the disclosures of which are incorporated by reference.

### BACKGROUND

The present disclosure is directed to modular components for prosthetic limbs and, more particularly, to modular shock absorbers for selective interconnection between components of prosthetic limbs.

### SUMMARY

Exemplary embodiments include a modular shock absorber for a prosthetic limb which may be selectively interconnected between a socket for receiving a residual limb and other prosthetic limb components. An exemplary embodiment may include a generally C-shaped body having a substantially planar first extension coupled to a prosthetic socket, a substantially planar second extension coupled to a prosthetic knee chassis, a substantially curved closed end connecting the first extension and the second extension, and/or further including a bias interposing the first extension and the second extension. Some exemplary embodiments may include first and second extensions that are horizontally and/or angularly offset relative to each other.

In a first aspect, a prosthetic leg may include a socket for receiving a patient's residual limb; an upright assembly; and a modular shock absorber selectively coupled between the socket and the upright assembly, the modular shock absorber including a first substantially planar extension coupled to a distal end of the socket, a second substantially planar extension coupled to a proximal end of the upright assembly, a closed end connecting the first extension and the second extension, and a bias interposing the first extension and the second extension. The modular shock absorber may be operative to allow the socket and the upright assembly to flex towards each at least under weight bearing forces.

In a detailed embodiment, the upright assembly may include a pylon coupled to a prosthetic foot. In a detailed embodiment, the closed end may be anteriorly disposed. In a detailed embodiment, the closed end may be posteriorly disposed.

In a detailed embodiment, the upright assembly may include a prosthetic knee assembly including a proximal segment and a distal segment, a pylon coupled to the distal segment, and a prosthetic foot coupled to a distal end of the pylon, and the second extension may be coupled to the proximal segment of the knee joint assembly. In a detailed embodiment, the closed end may be anteriorly disposed. In a detailed embodiment, the closed end may be posteriorly disposed.

In a detailed embodiment, the first substantially planar extension and the second substantially planar extension may be non-parallel such that respective planes of the first substantially planar extension and the second substantially planar extension intersect at a non-zero angular offset. In a detailed embodiment, the first substantially planar extension may include a first coupling and the second substantially planar extension may include a second coupling, and the first coupling and the second coupling may be horizontally spaced apart by a non-zero horizontal offset.

In a detailed embodiment, the closed end may be substantially curved. In a detailed embodiment, the first substantially planar extension, the second substantially planar extension, and the closed end may be included in a generally C-shaped body of material. In a detailed embodiment, the generally C-shaped body of material may be a strong, lightweight and resilient material.

In an aspect, a modular shock absorber for connecting a prosthetic socket and a prosthetic limb component may include a substantially planar first extension including a first coupling for connecting to a distal end of the prosthetic socket; a substantially planar second extension including a second coupling for connecting to the prosthetic limb component; and a connecting section joining the first extension and the second extension to form a generally C-shaped body. The first coupling may be at least one of horizontally offset and angularly offset relative to the second coupling.

In a detailed embodiment, the first coupling may be located farther from the connecting section than the second coupling. In a detailed embodiment, the first extension may extend beyond the second extension in a direction away from the connecting section. In a detailed embodiment, a plane of the first extension and a plane of the second extension may intersect at an intersection, and the connecting section may interpose the first and second sections and the intersection.

In a detailed embodiment, a modular shock absorber may include a bias interposing the first extension and the second extension. In a detailed embodiment, the bias may be substantially spherical, the bias may be constructed from a resilient material.

In an aspect, a modular shock absorber for selective interconnection between a prosthetic socket and a prosthetic limb component may include a substantially planar first extension including a plurality of holes for coupling to a prosthetic socket; a substantially planar second extension including a plurality of holes for coupling to a prosthetic limb component; a curved section connecting the first extension and the second extension such that the first extension is angled with respect to the second section. The first extension, the second extension, and the curved section may form a substantially C-shaped body, and the first extension may extend beyond the second extension in a direction away from the curved section.

In a detailed embodiment, the first extension, the second extension, and the curved section may have a substantially uniform width. In a detailed embodiment, a modular shock absorber may include a bias interposing the first extension and the second extension. In a detailed embodiment, the bias may include a resilient substantially spherical body mounted between the first extension and the second extension. In a detailed embodiment, the first extension and the second extension may diverge from the curved section.

### BRIEF DESCRIPTION OF THE DRAWINGS

The detailed description refers to the following figures in which:

FIG. 1 is a side elevation view of an exemplary above-knee prosthetic limb including an exemplary modular shock absorber in an anterior orientation;

FIG. 2 is a side elevation view of an exemplary modular shock absorber;

FIG. 3 is a rear elevation view of an exemplary modular shock absorber;

FIG. 4 is a front elevation view of an exemplary modular shock absorber;

FIG. 5 is a plan view of an exemplary modular shock absorber;

FIG. 6 is a bottom view of an exemplary modular shock absorber;

FIG. 7 is a side elevation view of an exemplary above-knee prosthetic limb including an exemplary modular shock absorber in a posterior orientation;

FIG. 8 is a side elevation view of an exemplary below-knee prosthetic limb including an exemplary modular shock absorber in an anterior orientation; and

FIG. 9 is a side elevation view of an exemplary below-knee prosthetic limb including an exemplary modular shock absorber in a posterior orientation.

### DETAILED DESCRIPTION

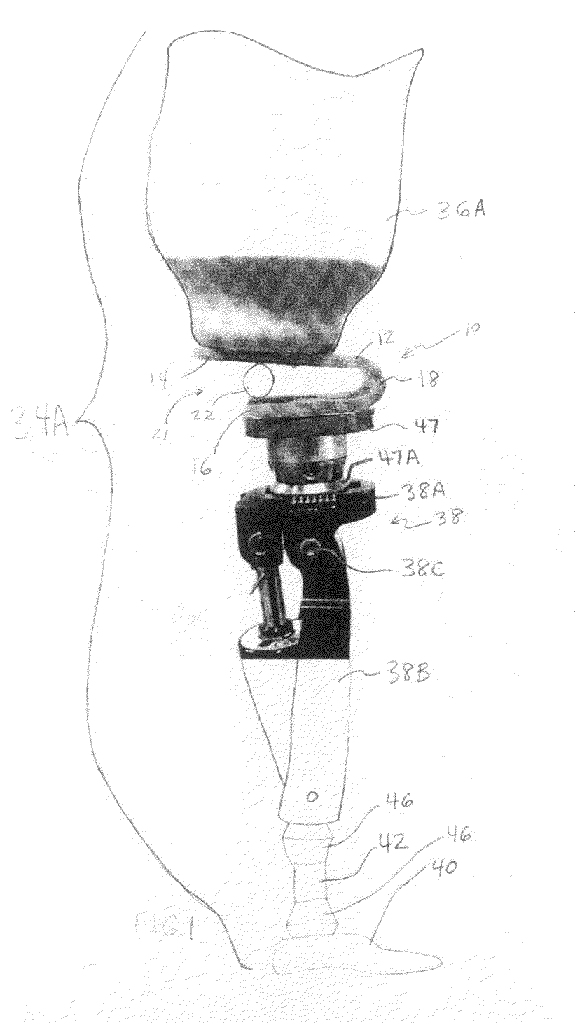

Referring to FIG. 1, an exemplary above knee (AK) prosthetic limb 34A may include a socket 36A for receiving a residual limb, a modular shock absorber 10, a pyramid coupling 47A, a pyramid receiver 47, a prosthetic knee chassis 38 (which may include a proximal segment 38A pivotably mounted to a distal segment 38B by a pivot 38C), a pylon 42 (which may include one or more pyramid receivers 46), and a prosthetic foot 40. An exemplary modular shock absorber 10 may include a generally C-shaped body 12 which may include a substantially planar first extension 14 and a substantially planar second extension 16. First extension 14 and second extension 16 may be joined by a substantially curved closed end 18 and may be interposed by a bias 22, which may be located near an open end 21 of the body 12. In FIG. 1, the modular shock absorber 10 is selectively (i.e., removably) installed in an anterior orientation (i.e., the closed end 18 is oriented substantially anteriorly).

In some exemplary embodiments, pyramid receivers 46, 47 may engage a corresponding pyramid mounted to an adjacent component. For example, referring to FIG. 1, pyramid coupling 47A (which may be mounted to the proximal segment 38A of prosthetic knee chassis 38) may engage pyramid receiver 47 mounted to second extension 16 of the shock absorber. Similarly, the distal end of the distal segment 38B of knee chassis 38 and/or the proximal surface of prosthetic foot 40 may include a pyramid coupling received by pyramid receivers 46 on pylon 42. Such pyramid couplings and pyramid receivers may be rotatably and/or laterally adjustable, may be integrated into their respective prosthetic limb components (including a modular shock absorber 10), and/or may be attached to their respective prosthetic limb components using fasteners (such as bolts or screws) and/or holes 24, 26, for example. In some exemplary embodiments, a modular shock absorber 10 may be mounted directly to the proximal segment 38A of a prosthetic knee chassis 38 without any interposing pyramid coupling components. Further, some exemplary embodiments may incorporate any known coupling devices to join the various components of the prosthetic limb.

In an exemplary embodiment, a shuttle lock may be mounted within the distal end of the socket 36A. The shuttle lock may receive a plunger pin extending from a distal end of a sleeve on the patient's residual limb. In some exemplary embodiments, the shuttle lock may comprise a PDI Xtreme® suspension lock, which is available from Prosthetic Design Incorporated of Clayton, Ohio.

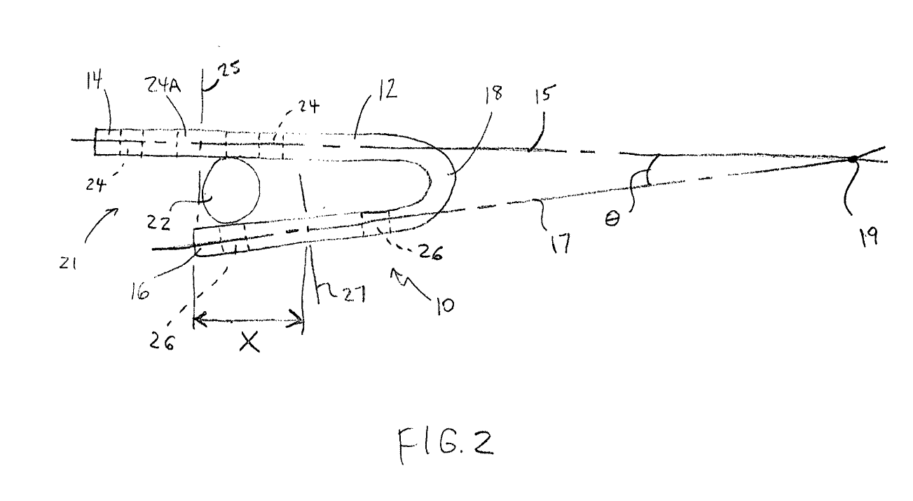

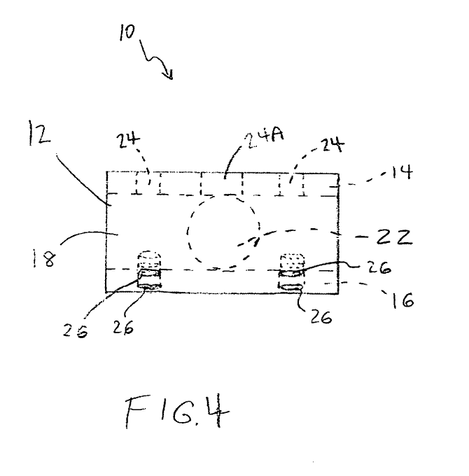

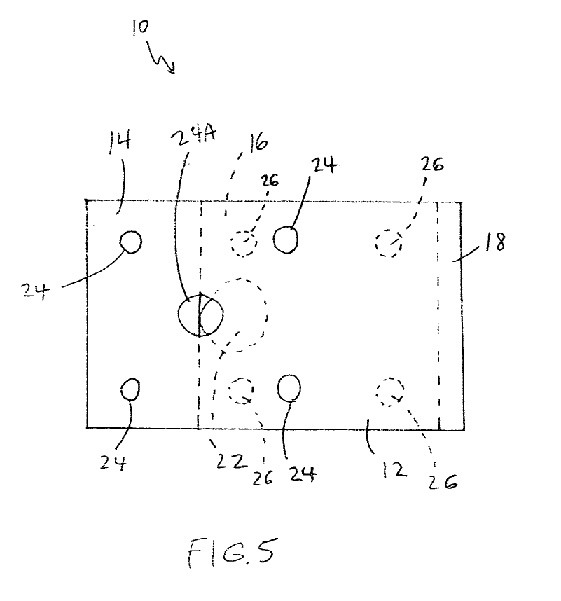

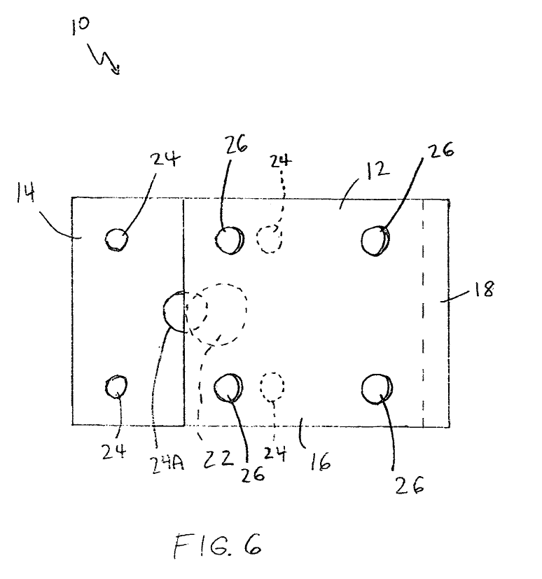

Referring to FIGS. 2-6, an exemplary modular shock absorber 10 may include a body 12 which may be constructed from a strong, lightweight, and resilient material, such as carbon fiber, composite resin, aluminum, titanium, and/or fiberglass. Body 12 may be formed from as a sheet of such material folded upon itself generally in the shape of a C to provide the first extension 14, the second extensions 16, and the closed end 18 as a substantially monolithic component.

In an exemplary embodiment, bias 22 may be constructed from a generally rubber-like material, such as a thermoplastic Elastomer, urethane, silicone, etc. For example, bias 22 may be constructed from Santoprene™ thermoplastic elastomer, which is a mixture of of EPDM rubber (ethylene propylene diene M-class rubber) and polypropylene and which is typically available in hardness grades from 35 Shore A to 60 Shore D.

In an exemplary embodiment, bias 22 may be substantially spherical and/or may have a diameter of approximately ⅜″ or more. Bias 22 may be retained in position near open end 21 between first extension 14 and second extension 16 by any manner of known mechanical and/or chemical couplings/fasteners such as, for example and without limitation, providing grooves on the bias 22 for mating with complementary ribs on the first extension 14 and/or second extension 16, or providing concave depressions in the first extension 14 and/or second extension 16 for receiving portions of the bias 22. In some exemplary embodiments, bias 22 may include one or more springs, and/or alternate shapes such as short cylinders.

A set of exemplary biases 22 may constructed from compositions having a variety of durometers. A prosthetist may select a bias 22 having appropriate characteristics from a plurality of available biases 22; thus, an exemplary modular shock absorber 10 may be adjustable based upon the needs of the patent.

In an exemplary embodiment, referring to FIGS. 2-6, a coupling associated with the first extension 14 may include one or more holes 24 (e.g., a set of four tapped holes 24) for receiving fasteners (such as threaded screws) extending from a proximal prosthetic limb component such as the socket 36A. Similarly, a coupling associated with the second extension 16 may include one or more holes 26 (e.g., a set of four through-holes 26) for receiving fasteners extending from a distal prosthetic limb component such as the pyramid coupling 47A. In various exemplary embodiments, any of the holes 24, 26 may be threaded (e.g., by use of a metal insert) or smooth, and any of the holes 24, 26 may be countersunk and/or counterbored to accommodate a fastener head, for example. It is also within the scope of the disclosure to provide more than one set or pattern of the holes 24 in first extension 14 and/or the holes 26 in second extension 16 for additional flexibility in coupling the modular shock absorber 10 to various prosthetic limb components. Some exemplary modular shock absorbers 10 may include additional features, such as hole 24A for accommodating the distal end of a plunger pin extending through the distal end of socket 36A.

As shown in FIG. 2, first extension 14 and second extension 16 may be non-parallel. For example, a plane 15 of first extension 14 may intersect a plane 17 of second extension 16 at an intersection 19. The angle at which plane 15 of first extension 14 and plane 17 of second extension 16 intersect may be referred to as the angular offset θ. In the exemplary embodiment shown in FIG. 2, first extension 14 and second extension 16 are angled such that the closed end 18 may interpose the first extension 14 and the second extension 16 and the intersection 19. Stated another way, the intersection 19 may be located nearest the closed end 18 of the body 12.

In some exemplary embodiments, first extension 14 and second extension 16 may be parallel or substantially parallel. In such embodiments, plane 15 and plane 17 may not intersect or may intersect at such a distance from body 12 that they are substantially parallel. In addition, some exemplary embodiments may be constructed such that the closed end 18 does not interpose the first extension 14 and the second extension 16 and the intersection 19. Stated another way, the intersection 19 may be located nearest the open end of the body 12.

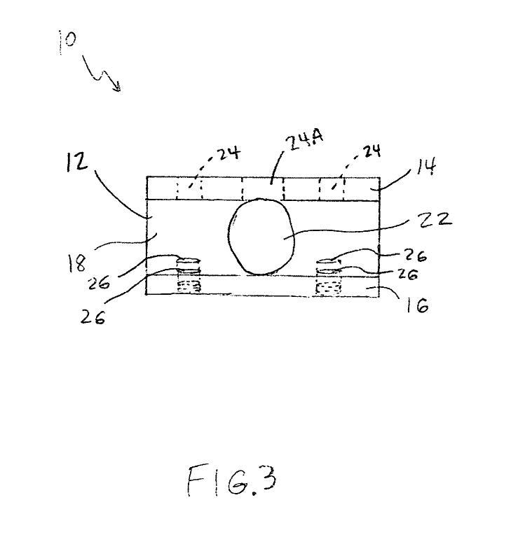

Also as shown in FIG. 2, a centerline 25 of a coupling associated with first extension 14 (e.g., holes 24) and a centerline 27 of a coupling associated with second extension 16 (e.g., holes 26) may be spaced in an anterior-posterior direction by horizontal offset X. FIGS. 3-6 are rear elevation, front elevation, plan, and bottom views of an exemplary modular shock absorber 10.

In use, during ambulation, the first extension 14 and the second extension 16 may flex towards each other at the open end 21, generally pivoting about closed end 18, during heel strike or another weight-bearing event. The bias 22 may absorb energy during such weight-bearing events, and may also provide a restorative force to help return the first extension 14 and the second extension 16 to their respective unloaded positions upon the weight-bearing event ending.

When installed in an anterior orientation in an exemplary AK prosthetic limb 34A as shown in FIG. 1, a modular shock absorber 10 may provide stance flexion. The present disclosure contemplates that stance flexion is a term used in the prosthetic industry to describe a feature on a knee-shin system that allows an amputee to exhibit “bent knee walking.” More simply, the stance flexion feature allows the knee axis to bend slightly under weight bearing without comprising the stability of the knee. Stance flexion allows a certain degree of knee flexion at heel strike, which may allow an amputee with a prosthetic limb to more closely mimic the normal gait patterns of non-amputees. In addition, an exemplary modular shock absorber 10 may reduce the forces felt by the amputee's residual limb during ambulation.

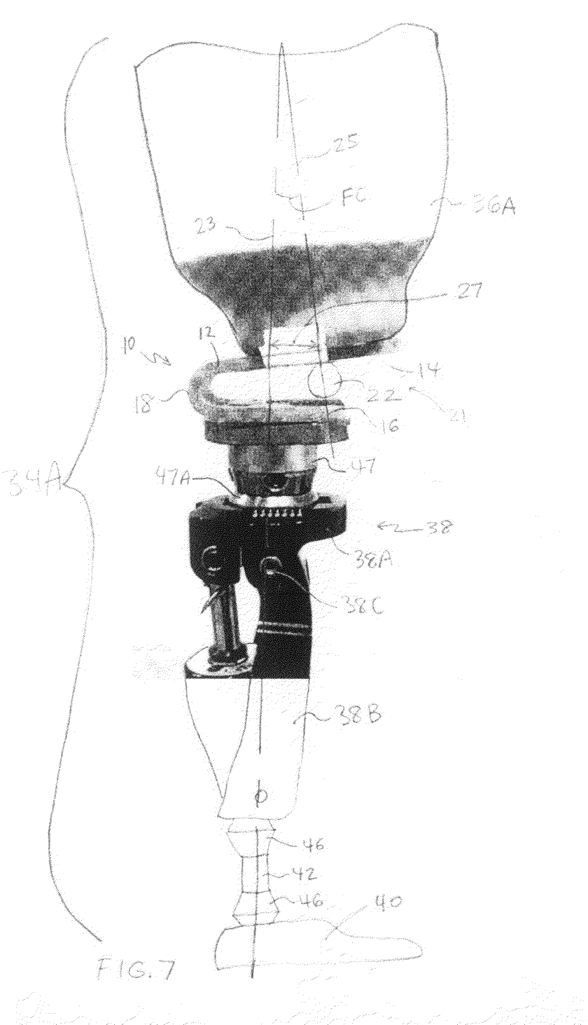

FIG. 7 is a side elevation view of an exemplary AK prosthetic limb 34A including an exemplary modular shock absorber 10 arranged in a posterior orientation (i.e., the closed end 18 is oriented substantially posteriorly). In this configuration, an exemplary modular shock absorber 10 may be utilized to at least partially compensate for a patient's hip flexion contracture. The present disclosure contemplates that hip flexion contracture may be defined as an angle FC between a body weight line 23 perpendicular to the supporting surface and a bisector line 25 of a flexed above-knee (AK) residual limb. The flexion contracture may cause the distal end of the residual limb to be offset anteriorly from the body weight line 23. See, e.g., U.S. Pat. No. 6,991,658, which is incorporated by reference. The anterior offset 27 of the distal end of the socket 36A may be approximately equal to the sine of the flexion contracture angle FC multiplied by the length from the patient's trochanter to the distal end socket 36A. An exemplary modular shock absorber 10 may be constructed (or selected from among a plurality of off-the-shelf modular shock absorbers) such that the angular offset θ and/or horizontal offset X at least partially assist a prosthetist in accommodating the flexion contracture angle FC and/or anterior offset 27. In addition, an exemplary modular shock absorber 10 may reduce the forces felt by the amputee's residual limb during ambulation.

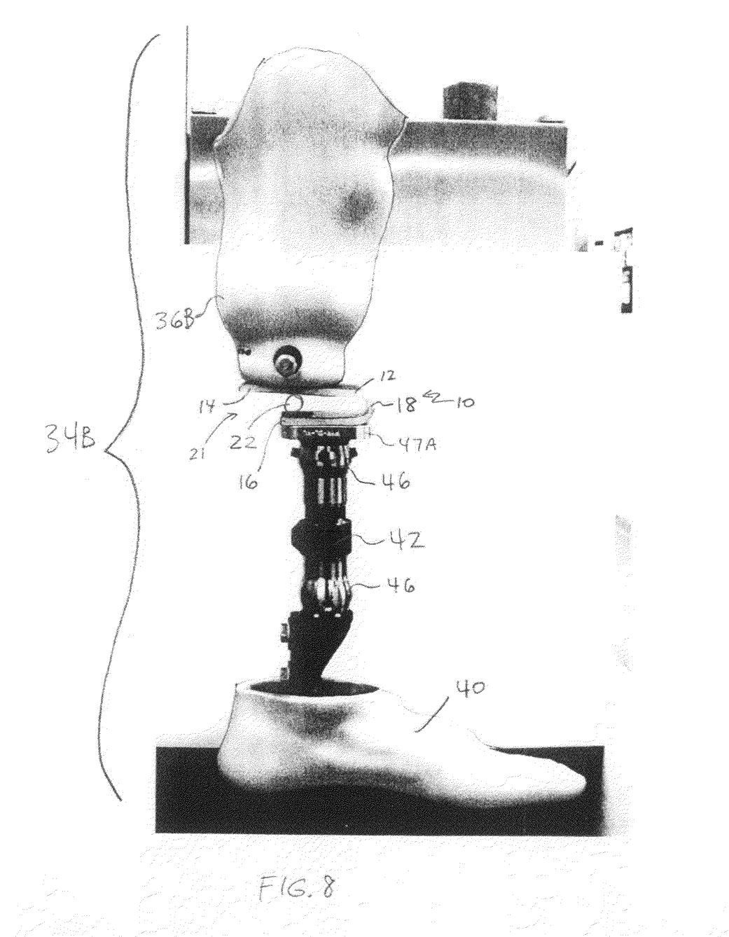

FIG. 8 is a side elevation view of an exemplary BK prosthetic limb 34B including an exemplary modular shock absorber 10 arranged in an anterior orientation between a socket 36B and pyramid coupling 47A, which is connected to a pylon 42 and a prosthetic foot 40. In this configuration, an exemplary modular shock absorber 10 may at least partially compensate for a patient's knee flexion contracture. In addition, an exemplary modular shock absorber 10 may reduce the forces felt by the amputee's residual limb during ambulation.

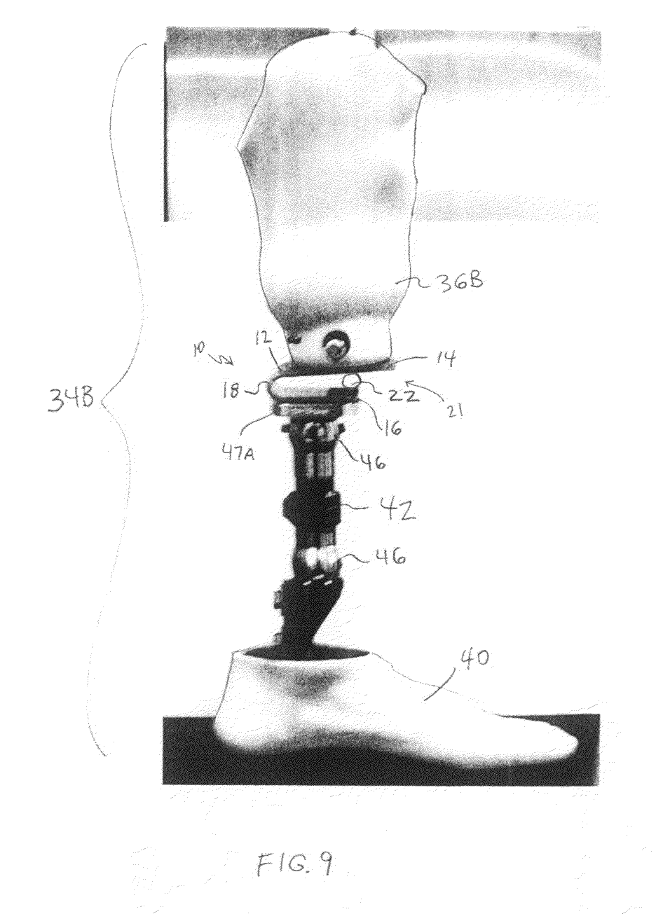

FIG. 9 is a side elevation view of an exemplary BK prosthetic limb 34B including an exemplary modular shock absorber 10 arranged in a posterior orientation between a socket 36B and a pyramid coupling 47A, which is connected to a pylon 42 and a prosthetic foot. In addition, an exemplary modular shock absorber 10 may reduce the forces felt by the amputee's residual limb during ambulation. An exemplary shock absorber 10 in this configuration may allow the knee to flex slightly under weight bearing at mid-stance without compromising the overall stability of the prosthetic device.

In some exemplary embodiments, first extension 14 and/or second extension 16 may include one or more access holes for installing or tightening fasteners associated with the other of the first extension 14 and second extension 16. For example, first extension 14 may include one or more access holes generally aligned with one or more of holes 26 in second extension 16 to allow installation and/or tightening of fasteners in holes 26.

In some exemplary embodiments, body 12 may have a non-uniform width. For example, the closed end 18 of the body 12 may include one or more notches, cutouts, or the like extending vertically therethrough to augment flexibility of the first extension 14 relative to the second extension.

Some exemplary embodiments may include a hyperextension limiting element, such as a strap connecting the first extension 14 and the second extension 16 near the open end 21. Such a device may prevent the modular shock absorber 10 from hyperextending (i.e., the ends of the first extensions 14 and the second extension 16 pivoting apart from each other too far). Such a limitation on hyperextension may also be provided by a mechanical linkage extending through the bias 22 and connecting the first extension 14 and the second extension 16.

While exemplary embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages of the may exist even though they may not have been explicitly discussed herein.

What is claimed is:

1. A prosthetic leg comprising:
a socket for receiving a patient's residual limb;
an upright assembly; and
a modular shock absorber selectively coupled between the socket and the upright assembly, the modular shock absorber including
a first substantially planar extension coupled to a distal end of the socket,
a second substantially planar extension coupled to a proximal end of the upright assembly,
a closed end connecting the first extension and the second extension, and
a bias interposing the first extension and the second extension;
wherein the modular shock absorber is operative to allow the socket and the upright assembly to flex towards each at least under weight bearing forces.

2. The prosthetic leg assembly of claim 1, wherein the upright assembly includes a pylon coupled to a prosthetic foot.

3. The prosthetic leg assembly of claim 2, wherein the closed end is anteriorly disposed.

4. The prosthetic leg assembly of claim 2, wherein the closed end is posteriorly disposed.

5. The prosthetic leg assembly of claim 1, wherein the upright assembly includes a prosthetic knee assembly including a proximal segment and a distal segment, a pylon coupled to the distal segment, and a prosthetic foot coupled to a distal end of the pylon; and wherein the second extension is coupled to the proximal segment of the knee joint assembly.

6. The prosthetic leg assembly of claim 5, wherein the closed end is anteriorly disposed.

7. The prosthetic leg assembly of claim 5, wherein the closed end is posteriorly disposed.

8. The prosthetic leg assembly of claim 1, wherein the first substantially planar extension and the second substantially planar extension are non-parallel such that respective planes of the first substantially planar extension and the second substantially planar extension intersect at a non-zero angular offset.

9. The prosthetic leg assembly of claim 1, wherein the first substantially planar extension includes a first coupling and the second substantially planar extension includes a second coupling; and wherein the first coupling and the second coupling are horizontally spaced apart by a non-zero horizontal offset.

10. The prosthetic leg assembly of claim 1, wherein the closed end is substantially curved.

11. The prosthetic leg assembly of claim 1, wherein the first substantially planar extension, the second substantially planar extension, and the closed end are included in a generally C-shaped body of material.

12. The prosthetic leg assembly of claim 11, wherein the generally C-shaped body of material is a strong, lightweight and resilient material.

13. A modular shock absorber for connecting a prosthetic socket and a prosthetic limb component, the modular shock absorber comprising:
a substantially planar first extension including a first coupling for connecting to a distal end of the prosthetic socket;
a substantially planar second extension including a second coupling for connecting to the prosthetic limb component; and
a connecting section joining the first extension and the second extension to form a generally C-shaped body;
wherein the first coupling is at least one of horizontally offset and angularly offset relative to the second coupling.

14. The modular shock absorber of claim 13, wherein the first coupling is located farther from the connecting section than the second coupling.

15. The modular shock absorber of claim 14, wherein the first extension extends beyond the second extension in a direction away from the connecting section.

16. The modular shock absorber of claim 13 wherein a plane of the first extension and a plane of the second extension intersect at an intersection; and wherein the connecting section interposes the first and second sections and the intersection.

17. The modular shock absorber of claim 13, further comprising a bias interposing the first extension and the second extension.

18. The modular shock absorber of claim 17, wherein the bias is substantially spherical; and wherein the bias is constructed from a resilient material.

19. A modular shock absorber for selective interconnection between a prosthetic socket and a prosthetic limb component, the modular shock absorber comprising:
a substantially planar first extension including a plurality of holes for coupling to a prosthetic socket;
a substantially planar second extension including a plurality of holes for coupling to a prosthetic limb component;
a curved section connecting the first extension and the second extension such that the first extension is angled with respect to the second section,
wherein the first extension, the second extension, and the curved section form a substantially C-shaped body; and
wherein the first extension extends beyond the second extension in a direction away from the curved section.

20. The modular shock absorber of claim 19, wherein the first extension, the second extension, and the curved section have a substantially uniform width.

21. The modular shock absorber of claim 19, further comprising a bias interposing the first extension and the second extension.

22. The modular shock absorber of claim 21, wherein the bias includes a resilient substantially spherical body mounted between the first extension and the second extension.

23. The modular shock absorber of claim 19, wherein the first extension and the second extension diverge from the curved section.
